(19) United States
(12) Patent Application Publication
(10) Pub. No.: US 2023/0313258 A1
(43) Pub. Date: Oct. 5, 2023

(54) CROSS-LINIKING METHODS

(71) Applicant: The University of Bath, Bath Somerset (GB)

(72) Inventor: Jody Michael MASON, Bath Somerset (GB)

(73) Assignee: The University of Bath, Bath Somerset (GB)

(21) Appl. No.: 18/002,553

(22) PCT Filed: Jun. 23, 2021
(86) PCT No.: PCT/EP2021/067255

§ 371 Date: Dec. 20, 2022

(30) Foreign Application Priority Data
Jun. 25, 2020 (GB) 2009710.1

(51) Int. Cl. C12P 21/02; C07K 14/47

(52) U.S. Cl. CPC C12P 21/02; C07K 14/4703

(57) ABSTRACT

Disclosed are methods and kits for producing a conformationally constrained peptide, such as a helix constrained peptide, in a cell. In some cases, the methods comprise contacting a cell comprising an intracellularly-localised recombinant peptide with a cross-linker and culturing the cell in the presence of the cross-linker, wherein the cross-linker forms thioether cross-links with at least two derivatisable amino acids located at anchoring positions in the recombinant peptide. The methods and kits and cells find application, for example, in the identification of inhibitors that can be used to disrupt protein-protein interactions.

This application claims priority from GB2009710.1 filed 25 Jun. 2020, the contents and elements of which are herein incorporated by reference for all purposes.

### FIELD OF THE INVENTION

The present invention relates to a method of producing a conformationally constrained peptide, such as a helix constrained peptide, in a cell. In particular, the present invention relates to methods that involve producing the conformationally constrained peptide in the cell and carrying out intracellular screening assays for example to assay whether the conformationally constrained peptide is able to inhibit association between a first and second candidate binding partner present in the cell. The methods of the invention find application, for example, in the identification of inhibitors that can be used to disrupt protein-protein interactions.

### BACKGROUND

Protein-protein interactions (PPIs) are fundamentally important for the function of a huge variety of biological processes. Molecules that are capable of specifically modifying PPIs are highly sought after for use as probes and therapeutic agents that can potentially be used to inhibit PPI. Unfortunately, PPIs, especially those that occur intracellularly, have proven challenging targets for conventional drug compounds such as small molecules and biologics.

The particular residues present on the surface of a protein that are responsible for PPIs are often associated with protein secondary structure motifs, such as alpha-helix, beta-sheets and beta-turns. Of note, alpha-helices are thought to comprise approximately 60% of all secondary structures in protein complexes (Jochim and Arora, 2010). Additionally, alpha-helices have been shown to mediate a large number of key therapeutically relevant PPI interfaces, of which 60% bind to one face of the helix (Raj et al., 2013). Accordingly, some investigators have turned to peptides that contain stabilised alpha-helices as an approach to identify inhibitors of PPIs.

Constraining peptides in a helical conformation has been reported to confer benefits that include entropic preorganization for effective binding, enhanced protease resistance, stability in cells, increased cellular uptake, enhanced biophysical properties and are anticipated to bind their targets with higher potency in comparison to wild-type peptide sequences (Azzarito et al. 2013). As a result, peptides that contain constrained alpha-helices (also termed “helix-constrained peptides”) have been of great interest for identifying PPI inhibitors (Robertson and Spring, 2018).

A number of chemical strategies have been established to contain peptides in a particular conformations. For example, carbon-carbon bonds, disulfide bridges, lactam linkages, oxime, hydrazones, triazoles and thioether bonds have all been applied to connect (“staple”) two side-chains of native or non-native anchoring residues in a peptide. Among the naturally occurring amino acid residues, cysteine is considered particularly convenient as a conjugation target for cross-linkers (Fairlie & Dantas de Araujo, 2016). Methods for producing conformationally constrained peptides through cysteine residues typically involve introducing cysteines into particular anchoring positions within the peptide and the subsequent formation of thioester bonds between their side-chain sulfhydryl groups with an appropriate bis-functional cross-linker. The cross-linking step is typically carried out in vitro, often under reducing conditions. Examples of suitable cross-linkers that have been used in vitro to generate helix-constrained peptides by connecting two cysteine residues are described in Fairlie & Dantas de Araujo, 2016, Jo et al., 2012 and Timmerman et al. 2005.

It is, however, very difficult to predict whether if a given peptide sequence will tolerate a conformational constraint and almost impossible to know from rationale design alone whether a conformationally constrained peptide will be able to modulate intracellular PPIs. Whilst approaches such as phage display have been utilised to synthesise and chemically modify large combinatorial libraries of constrained peptides in vitro (e.g. as described in Heinis & Winter, 2015), downstream screening of individual library members is still required to determine whether the conformationally constrained peptide will be able to disrupt intracellular PPIs.

Thus, there remains a need for methods that are able to conformationally constrain peptides, in particular methods that will simplify the process for identifying conformationally constrained peptides that are capable of inhibiting PPIs.

The present invention has been devised in light of the above considerations.

### DISCLOSURE OF THE INVENTION

The present inventors made the surprising discovery that it is possible to introduce an intra-molecular cross-link into a peptide to produce a conformationally constrained peptide whilst it remains inside the cell. Since the helix-constrained peptide is present within the cell, the cells can immediately be used for subsequent intracellular screening assays to determine whether the conformationally constrained peptide is able to disrupt PPIs. Accordingly, the present method offers a “one-pot reaction” where the same cell can be used to produce the helix-constrained peptide and test for whether it is able to disrupt intracellular PPIs. This is believed to represent a more efficient process than those methods disclosed in the prior art where peptides are first chemically modified outside the cell (e.g. in solution or whilst expressed on the surface of a phage) to produce the conformationally constrained peptide and then subsequently introducing this peptide into a cell to confirm intracellular activity.

Thus, in one aspect the present invention provides a method of producing a conformationally constrained peptide in a cell, the method comprising:
i) providing a cell containing a recombinant peptide, wherein the recombinant peptide comprises at least two derivatisable amino acid residues located at anchoring positions, each derivatisable amino acid comprising a reactive thiol group;
ii) contacting the cell with a cross-linker, wherein the cross-linker is capable of reacting with said reactive thiol groups; and
iii) culturing the cell in the presence of the cross-linker, such that the cross-linker forms thioether cross-links with the at least two derivatisable amino acids, thereby producing the conformationally constrained peptide in the cell.

The recombinant peptide is expressed and remains entirely localised within the cell, i.e. within the cell cytoplasm, during production of the conformationally constrained peptide. This means that screening assays that determine whether the conformationally constrained peptide is able to modulate (e.g. inhibit) protein-protein interactions (PPIs) between a first and second candidate binding partner can be carried out in the same cell that is used for production of the conformationally constrained peptide. Suitable intracellular assays for identifying whether the conformationally constrained peptide is able to inhibit PPIs are described in more detail below.

Accordingly, in some embodiments the cell further comprises a first and second candidate binding partner and the method further comprises assaying whether the conformationally constrained peptide is able to inhibit association between the first and second candidate binding partner. Assaying for whether the conformationally constrained peptide is able to inhibit association between the first and second candidate binding partner may involve determining whether it is able to modulate (e.g. increase or decrease) the activity and/or expression of a reporter protein. In some embodiments, the conformationally constrained peptide may be classed as an inhibitor of the PPI between the first and second candidate binding partners if it is able to modulate activity and/or expression of the reporter protein.

In these assays, activity of the reporter protein is controlled by the association between a first and second candidate binding partners. Reporter protein activity may be directly controlled by the association of the first and second candidate binding partners. For example, the first candidate binding partner is linked (e.g. fused) to a first fragment of the reporter protein and the second candidate binding partner is linked (e.g. fused) to the second fragment of the reporter protein, where association (e.g. dimerisation) of the first and second candidate binding partners reconstitutes reporter protein activity (i.e. brings the first and second fragments of the reporter protein into close enough proximity for activity to be established). In cases where association of the first and second candidate binding partners forms the reporter protein, the additional presence in the cell of a peptide that inhibits association between the first and second candidate binding partners will decrease activity of the reporter protein.

Alternatively, reporter activity may be indirectly controlled by the association of the first and second candidate binding partners. For example, association of the first and second candidate binding partners forms a DNA-binding complex that binds a binding site in a nucleic acid encoding the reporter protein, wherein binding to the binding site inhibits or promotes expression of the reporter protein. In cases where association of the first and second candidate binding partners inhibits expression of the additional presence in the cell of a peptide that inhibits association between the first and second candidate binding partners will result in increased expression of, and hence increased activity of, the reporter protein. In cases where association of the first and second candidate binding partners promotes expression of the reporter protein, the additional presence in the cell of a peptide that inhibits association between the first and second candidate binding partners will result in decreased expression of, and hence decreased activity of, the reporter protein.

The cross-linker used in the methods of the invention is capable of accessing the cytosol of the cell in order to react with the reactive thiol groups present in the intracellularly-localised recombinant protein.

In some embodiments, the cross-linker is a compound of formula 1:

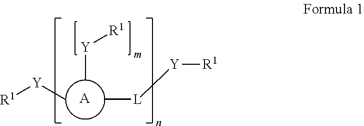

wherein
n is an integer selected from 1 to 3;
m is an integer selected from 0 to 2;
A is selected from C₂₋₆-alkenylene, C₅₋₁₂-arylene and C₅₋₁₂-heteroarylene;
Y is a covalent bond, C₁₋₆-alkylene or —N(H)C(═O)CH₂—;
R¹ is selected from Cl, Br, I, or F; and
each L is independently selected from —C(═O)—, —C≡C—, —N═N—, C₁₋₆-alkylene and a covalent bond.

The R¹ groups provide reactive groups (e.g. leaving groups) for reaction with the cysteine. The A groups provide the linkers with structures suitable for conformationally constraining a peptide in a call when cross inked via the two derivatisable amino acid residues. For example, the A group may be conformational constrained into a geometry suitable for linking the two derivatisable amino acid residues.

In preferred embodiments, the cross-linker is a compound of formula 2aa:

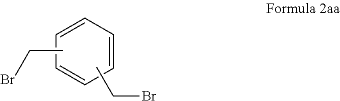

In particularly preferred embodiments, the cross-linker is 1,2 dibromomethylbenzene, 1,3 dibromomethylbenzene, or 1,4 dibromomethylbenzene. In even more preferred embodiments, the cross-linker is 1,3 dibromomethylbenzene (DBMB) having the formula:

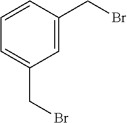

The crosslinker forms thioether cross-links with the at least two derivatisable amino acids such that the conformationally constrained peptide may comprise the structure:

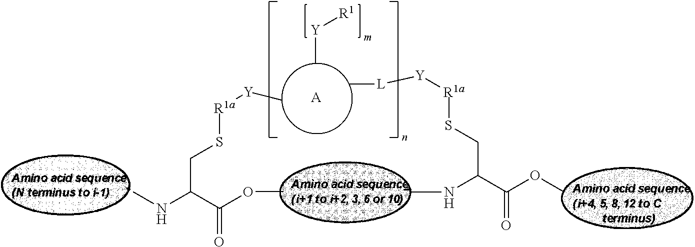

Y, L, R¹, n, m and A are as defined for formula 1. R^{1a} represents a bond or Ch2-CH2- linker derived from the appropriate R1 group in formula 1.

In embodiments where the cross-linker is DBMB, the conformationally constrained peptide may comprise the structure:

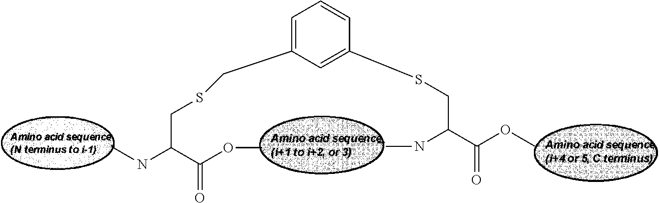

The inventors found that bacterial cells grew in the presence of DBMB, demonstrating the cross-linker is not toxic and is therefore suitable for use in the methods described herein. Furthermore, evidence is provided herein that when cells are grown in the presence of DBMB, the cross-linker is able to move from the cell culture media into the cells and forms cross-links with cysteine residues within a recombinantly expressed peptide located within the cells.

In another aspect, the present invention provides a kit comprising
i) a nucleic acid encoding a recombinant peptide, wherein the recombinant peptide comprises at least two derivatisable amino acid residues located at anchoring positions, each derivatisable amino acid comprising a reactive thiol group; and
ii) a cross-linker, wherein the cross-linker is capable of reacting with said reactive thiol groups, such that the cross-linker is capable of forming thioether cross-links with the at least two derivatisable amino acids.

In another aspect, the present invention provides the use of a cross-linker for producing a conformationally constrained peptide in a cell, the use comprising:
i) providing a cell containing a recombinant peptide, wherein the recombinant peptide comprises at least two derivatisable amino acid residues located at anchoring positions, each derivatisable amino acid comprising a reactive thiol group;
ii) contacting the cell with the cross-linker, wherein the cross-linker is capable of reacting with said reactive thiol groups; and
iii) culturing the cell in the presence of the cross-linker, such that the cross-linker forms thioether crosslinks with the at least two derivatisable amino acids, thereby producing the conformationally constrained peptide in the cell/

Recombinant Peptides and Expression

As used herein, a “recombinant peptide” is a peptide that is expressed within a cell which does not naturally express the peptide. Typically the recombinant peptide is produced by recombinant DNA technology.

The recombinant peptide is expressed and remains entirely localised within the cell, i.e. within the cell cytoplasm, during production of the conformationally constrained peptide (i.e., the peptide is “intracellularly-localised”). When the cell is contacted with the cross-linker the peptide is intracellularly localised and not, for example, expressed on the extracellular surface of the cell. Methods of determining whether the recombinant peptide are localised within the cell are known in the art. For example, cells expressing the peptide can be lysed and separated into different fractions using differential centrifugation allowing for the identification of the peptide in the cytosol fraction using standard techniques.

The recombinant peptide comprises at least two derivatisable amino acid residues located at anchoring positions within the recombinant peptide. The recombinant peptide may comprise two, three or four, five or six derivatisable amino acid residues, preferably two or three derivatisable amino acid residues, more preferably two derivatisable amino acid residues. As used herein a “derivatisable amino acid residue” refers to an amino acid residue (natural or non-natural) having a reactive side chain group. The derivatisable amino acid residues preferably comprise a reactive thiol group (—SH) or a reactive selenol group (—SeH). In embodiments where the derivatisable amino acid residues comprise a reactive selenol group, the amino acid residue may be selenocysteine. Methods for incorporating selenocysteine into peptides via codon reprogramming are known in the art, for example as described in Craik, 2012.

Preferably the derivatisable amino acid residues comprise a reactive thiol group. Each derivatisable amino acid residues may be a cysteine (i.e. L-cysteine) or a cysteine derivative such as D-cystine or homocysteine. Preferably all the derivatisable amino acid residues present in the recombinant peptide are cysteine residues. Methods for engineering derivatisable amino acid residues (e.g. cysteines) into pre-determined anchoring positions in the recombinant peptide are well known in the art.

In particular embodiments, the conformationally constrained peptide is a helix-constrained peptide. The term “helix-constrained peptide” is intended to mean a peptide having at least one chemical modification that results in a cross-link between two derivatisable amino acid residues (e.g. cysteines) located at anchoring positions within the peptide in order to produce a stabilised alpha-helix. In particular, the peptide is chemically modified with a cross-linker that forms thioether crosslinks (i.e. C-S-C) with the at least two derivatisable amino acids.

The presence of a stabilised alpha-helix can be determined using methods such as circular dichroism spectroscopy for an alpha-helix, for example as described in Jo et al. (2012). Circular dichroism be used to measure a helicity increase, i.e. linear to cyclic. In situations where the cross-linking occurs through the formation of a disulphide bridge between two thiol groups, such as between two cysteine residues, the presence of a stabilised alpha-helix can also be determined using an assay that determining if thiols in the sample are free or conjugated. For example, free thiols can be assayed via reaction with Ellman's reagent (5,5′-dithiobis(2-nitrobenzoic acid; DTNB) (Sigma)) and monitoring absorbance at 412 nm. The presence of the cross-link in the conformationally-constrained peptide can also be determined by assaying for whether the mass of the peptide (e.g. via mass spectrometry) increases by the expected amount upon formation of the cross-link. For example, where the cross-linker is 1,3 dibromomethylbenzene, formation of the thioether cross-link between two cysteines in the peptide would be expected to increase the mass of the peptide by about 102 Da. The presence of a cross-link in a peptide that contains an aromatic group (e.g. as in DBMB) can also be determined by measuring absorbance at 200-300 nm of the purified peptide following cross-linking, as demonstrated in the examples.

In some embodiments, the method further comprises carrying out an assay for the presence of the helix-constrained peptide.

Generally, the anchoring positions are chosen such that the cross-link extends across the length of one, two or three helical turns (i.e. about 3-3.6 amino acid residues, about 7 amino acids residues or about 11 amino acid residues). Accordingly, amino acids positioned at i and one of: i+3, i+4, i+7 and i+11 in the recombinant peptide are ideal candidates for anchoring positions. Thus, for example, where a peptide has the sequence . . . X1-X2-X3-X4-X5-X6-X7-X8-X9-X10-X11-X12-X13 . . . , and the amino acid X is independently selected for each position, cross-links between X1 and X4 (i and i+3), or between X1 and X5 (i and i+4), or between X1 and X8 (i and i+7), or X1 and X12 (i and i+11) are useful as are cross-links between X2 and X5 (i and i+3), or between X2 and X6 (i and i+4), or between X2 and X9 (i and i+7), or between X2 and X13 (i and i+11) etc. The use of multiple cross-links (e.g., 2, 3, 4 or more) is also contemplated. In preferred embodiments, the anchoring positions are positions i and i+4 in the amino acid sequence of the recombinant peptide.

In preferred embodiments, the only amino acid residues that comprise a reactive thiol group in the recombinant peptide (or in the polypeptide comprising the recombinant peptide, if applicable) are the derivatisable amino acid residues located at the anchoring positions. This reduces the possibility of cross-links between residues that are not located at anchoring positions spanning the helical turn(s), which are unlikely to form helix-constrained peptides. Typically, the other amino acid residues located in the recombinant protein are independently any amino acid that does not comprise a reactive thiol group.

Thus, for example, where a peptide has the sequence . . . X1-X2-X3-X4-X5-X6-X7-X8 . . . , and the derivatisable amino acid residues are located at positions X2 and X6 (i and i+4), positions X1, X3, X4, X5, X7 and X8 can independently be any amino acid that does not comprise a reactive thiol group.

In some embodiments, the recombinant peptide comprises the amino acid sequence X1-X2-X3-X4-X5, wherein X1 and X5 are the at least two derivatisable amino acid residues and wherein X2, X3 and X4 are independently any amino acid, optionally wherein X2, X3 and X4 do not comprise a reactive thiol group that is capable of reacting with the cross-linker.

In some embodiments, the recombinant peptide is expressed intracellularly from a nucleic acid. For example, the nucleic acid encoding the recombinant peptide may be an expression cassette (also termed a “recombinant peptide expression cassette”), which may be delivered to the cell, optionally as part of an expression vector, or may be incorporated into the genome of the cell.

Typically, an expression cassette comprises a promoter operably linked to a protein coding sequence.

The term “operably linked” includes the situation where a selected coding sequence and promoter are covalently linked in such a way as to place the expression of the protein coding sequence under the influence or control of the promoter. Thus a promoter is operably linked to the protein coding sequence if the promoter is capable of effecting transcription of the protein coding sequence. In some embodiments, the expression cassette may further comprise further components of a eukaryotic or prokaryotic gene, such as one or more selected from the a list consisting of: an intron, an enhancer, a silencer, a 5′ UTR, a 3′ UTR, and a regulator.

Any suitable promoter known in the art may be used in the expression cassette providing it functions in the cell type being used. For example, where the cell is a bacterial cell, expression may be under control of the lac operon. In such cases, the cell may also contain a lac repressor protein, whereby expression can be controlled by the introduction of isopropyl β-D-1-thiogalactopyranoside (IPTG). The promoter may be endogenous to the cell in which the method is being carried out. Where multiple expression cassettes are used, each coding sequence may be independently operably linked to its own promoter. Alternatively, the coding sequence for one or more of the expression cassettes may be operably linked to the same promoter.

The expression cassettes described herein may be part of one or more expression vector(s). An “expression vector” as used herein is a DNA molecule used for expression of foreign genetic material in a cell. Any suitable vectors known in the art may be used. Suitable vectors include plasmids, binary vectors, viral vectors and artificial chromosomes (e.g. yeast artificial chromosomes). Alternatively, the expression cassettes described herein may be incorporated into the genome of the cell.

The methods described herein may comprise delivering (or “administering”) one or more nucleic acids described herein to the cell. Molecular biology techniques suitable for administering nucleic acids and producing peptides such as the recombinant peptide described herein in cells are well known in the art, such as those set out in Sambrook et al., Molecular Cloning: A Laboratory Manual, New York: Cold Spring Harbor Press, 1989.

Techniques for expressing peptides such that they are localised intracellularly or extracellularly are well known in the art. Typically, if secretion from the cell is desired, the recombinant peptide will comprise a signal peptide at or near the N-terminus of the protein that function to localise the protein to a particular location outside the cytoplasm, e.g. secreted from the cell or inserted into the cell membrane. In the present invention, the recombinant peptides are localised intracellularly, i.e. they should remain in the cytoplasm. Accordingly, nucleic acid encoding the recombinant peptide should not encode a signal peptide that provides for secretion of the recombinant peptide outside the cytoplasm.

The method of the invention is expected to have use with genetically encoded peptide libraries. Genetically encoded peptide libraries are known and have been used in screening methods for identifying inhibitors of PPIs. See, for example, Mern et al. (2010). Briefly, such libraries are formed from libraries of nucleic acids, each of which encodes and is capable of directing expression of a different recombinant peptide. Accordingly, in embodiments that involve genetically encoded peptide libraries, the resulting recombinant peptides in the libraries can be designed to always contain the at least two derivatisable amino acid residues (e.g. cystine residues) at the anchoring positions, but the remaining amino acid residues in the peptide can be randomly selected (“scrambled”). Such genetically encoded peptidic libraries can be used with the method of the present invention to rapidly conformationally constrain and screen multiple different test recombinant peptides at the same time.

Thus, in some embodiments the cell is part of a plurality of cells comprising a library of recombinant peptides, the library of recombinant peptides comprising a mixture of recombinant peptides that differ in one or more of their amino acid residues outside the anchoring positions.

This method is applicable for polypeptides that contain the conformationally constrained peptide, allowing the conformationally constrained peptide to be screened to determine if it can disrupt PPIs in the context of the polypeptide. In this specification the term “peptide” is intended to mean molecules that contain between 2 and 50 amino acids and the term “polypeptide” is intended to mean molecules that are made up of more than 50 amino acids Thus, in some embodiments the cell contains a polypeptide, wherein the polypeptide comprises the recombinant peptide defined herein.

In some embodiments, the recombinant peptide is derived from a parent peptide one that has previously been identified as being able to inhibit association between a first and second candidate binding partner, or is suspected of being able to inhibit association between a first and second candidate binding partner. For example, the parent peptide may be suspected to be a PPI inhibitor based on a PCA assay. A recombinant peptide that is “derived from” a parent peptide typically comprises the amino acid sequence of the parent peptide but is modified to comprise the at least two derivatisable amino acid residues located at anchoring positions. The recombinant peptide may further comprise 1, 2, 3, 4, 5, 6, 7, 8, 9 or 10 amino acid alterations (e.g. substitutions, insertions or deletions) compared to the parent peptide, where these alterations are outside the anchoring positions.

For example, the parent peptide may be the peptide ‘FosW’ having the sequence ASLDELQAEIEQLEERNYALRKEIEDLQKQLEKLGAP (SEQ ID NO: 1). FosW was previously identified as able to dissociate the dimeric AP-1 transcription factor from its DNA binding site (Mason et al. 2006). Exemplary recombinant peptides that are derived from FosW are provided as follows (locations of the two derivatisable amino acid residues in each peptide are emphasised):

(SEQ ID NO: 2)
ASL [symbol] ELQ [symbol] EIEQLEERNYALRKEIEDLQKQLEKLGAP;
(SEQ ID NO: 3)
ASLDELQAEI [symbol] QLE [symbol] RNYALRKEIEDLQKQLEKLGAP;
(SEQ ID NO: 4)
ASLDELQAEIEQLEERN [symbol] ALR [symbol] EIEDLQKQLEKLGAP;
(SEQ ID NO: 5)
ASLDELQAEIEQLEERNYALRKEI [symbol] DLQ [symbol] QLEKLGAP;
and
(SEQ ID NO: 6)
ASLDELQAEIEQLEERNYALRKEIEDLQ [symbol] QLE [symbol] LGAP.

In some embodiments, the recombinant peptide comprises a heptad repeat. A heptad repeat is a structural motif labelled abcdefg, in which amino acid residues at position a and d are conserved. The amino acids residues at position a and dare typically hydrophobic, but in some cases can include polar amino acid residues (e.g. asparagine or lysine). The amino acids at positions a and d can be any one of the following amino acid residues: alanine (A), valine (V), leucine (L), isoleucine (I), phenylalanine (F), methionine (M), asparagine (N), threonine (T) and lysine (K). The conservation of hydrophobic residues alternatively three and four residues apart is close to the 3.6 amino acids per turn periodicity of a regular alpha-helix. Consequently, helices deriving from such repeating sequences exhibit distinct amphipathic character, with both hydrophobic and polar faces and the association of two helices via their hydrophobic faces drives coiled-coil formation. Recombinant peptides comprising heptad repeats therefore may be suitable peptides that are able to antagonise coiled-coil interactions such as those involved in the formation of basic leucine zippers (bZIPs).

In some embodiments where the recombinant peptide comprises a heptad repeat, the i and i+4 anchoring positions are located at positions b and f in the heptad repeat. Optionally positions a and d in the heptad repeat may comprise one of the following amino acid residues: alanine (A), valine (V), leucine (L), isoleucine (I), phenylalanine (F), methionine (M), asparagine (N), threonine (T) and lysine (K). For example, the peptides represented by SEQ ID NOs 2, 3, 4 and 5 all contain cysteines at positions b and f in the heptad repeat. Previous work has examined peptide libraries consisting of seven residue sequences that correspond to one heptad repeat of a coiled-coil motif (gabcdef) where positions b and f in the heptad repeat were constrained using KD lactamisation (Baxter et al., 2017; Lathbridge and Mason, 2019; Rao et al. 2013).

In other embodiments where the recombinant peptide comprises more than one (e.g. two) heptad repeats, the i and i+4 anchoring positions may be located at positions f of one heptad and position c of the following heptad. For example, the peptide represented by SEQ ID NO: 6 contains cysteines at position f of one heptad and position c of the following heptad.

Candidate Binding Partners and Screening Methods

The methods described herein may comprise assaying for whether the conformationally constrained peptide is able to inhibit association between the first and second candidate binding partner.

The candidate binding partners can be any peptidic molecules that associate with one another (or are expected to do so). The first and second binding partners may have an identical amino acid sequence (e.g. they may homodimerise with each other). Alternatively, the first and second binding partners may have different amino acid sequences (e.g. they may heterodimerise with each other).

In some embodiments, the candidate binding partners form a DNA-binding complex upon association. Suitable candidate binding partners that form a DNA-binding complex upon association include those of the basic helix-loop helix (bHLH) or bHLH leucine zipper (bHLH-Zip) transcription factor families. bHLH and bHLH-Zip transcription factors are exclusively eukaryotic proteins that bind to sequence-specific double-stranded DNA as homodimers or heterodimers to either activate or repress gene transcription.

Exemplary human bZIP transcription factor subfamilies, the nucleotide sequences of their binding sites and examples of proteins of these subfamilies are set forth in Table 1 below. The candidate binding partners may be or may comprise any of these human bZIP proteins For example, the first and second candidate binding partners may proteins of the Fos/Jun bZip family that form a DNA-binding complex upon association.

TABLE 1

| Human bZIP | | Nucleotide sequence(s) of | |
|---|---|---|---|
| subfamily | Exemplary bZIP protein | binding site | Name of binding site |
| PAP | PAP1, YAP1, YAP2, YAP3, | TTACGTAA | PAP/CREB-2/PAR |
| | YAP4, YAP5, YAP6, YAP7, | | |
| | Cap1 | | |
| CREB-2 | AFT4, mATFP4, ApCREB-2, | | |
| | hCREB2, acr1 | | |
| PAR | DBP, VBP/TEF, HLF, CES2, | | |
| | TEF | | |
| C/EBP | C/EBPα, C/EBPβ, C/EBPδ, | ATTGCGCAAT | CCAAT |
| | C/EBPε, C/EBPγ, CRP1, | | |
| | CRP2, CRP3, Ig/EBP, Iap, | | |
| | DDIT3 | | |
| Fos/Jun | cFos, FRA1 (FosL1), FRA2 | TGACTCA or TGAGTCA | TPA response |
| | (FosL2), cJun, JUNB, JUND, | | element (TRE) |
| | GCN4, BATF, BATF2, | | |
| | BATF3 | | |
| CREB | CREB1, ATF1, ATF2, ATF3, | TGACGTCA | CAMP response |
| | ARF5, ATFa, BBF-2, | | element (CRE) |
| | CREB3L1 | | |
| Maf | MafA, MafB, BACH1, | TGCTGA(G/C)TCAGCA and | Maf recognition |
| | BACH2 | TGCTGAG(C/C)GTCAGCA | element (MARE) |

In other examples, the candidate binding partners may form protein aggregates, or may be expected to do so. Protein aggregates are typically formed where multiple misfolded proteins accumulate and clump together and their presence is associated with a number of diseases, in particular neurodegenerative diseases such as Alzheimer's Disease (AD), Parkinson's disease (PD) and prion disease (also known as transmissible spongiform encephalopathy). In some embodiments, the presence of an aggregate of the candidate binding partners in a human patient is associated with a disease or other pathological condition, such as a neurodegenerative disease.

Examples of peptides and polypeptides that are capable of forming protein aggregates include those that are capable of aggregating to form amyloids, as well as those capable of aggregating to form amorphous or native-like deposits. In some embodiments, the candidate binding partners are amyloid-β (Aβ) peptides, α-synuclein (αS) polypeptides, tau proteins, or prion proteins.

In other examples, PPI between the candidate binding partners may associated with an intracellular signalling pathway. Numerous intracellular signalling pathways are associated with the interaction (which may be covalent or non-covalent) between one or more proteins, e.g. an enzyme such as a kinase. Accordingly, one of the candidate binding partners could be an enzyme such as a kinase and the other candidate binding partner be a protein that interacts (e.g. binds to) the enzyme. For example, guanine nucleotide exchange factors (GEFs) are proteins or protein domains that associate with small GTPases to induce catalytic activity of the GEF. Exemplary methodology for designing and producing peptides that can target and modulate helical PPIs associated with intracellular signalling pathways is provided in Yoo et al. 2020.

In some embodiments, the candidate binding partners are expressed intracellularly from one or more nucleic acids. For example, one or more nucleic acids encoding the candidate binding partners may be an expression cassette (also termed a “candidate binding partner expression cassette”), which may be delivered to the cell, optionally as part of an expression vector, or may be incorporated into the genome of the cell. Where the first and second candidate binding partners have an identical amino acid sequence, both binding partners may be expressed from the name nucleic acid.

Assaying for whether the conformationally constrained peptide is able to inhibit association between the first and second candidate binding partners may involve determining whether the conformationally constrained peptide is able to modulate activity and/or expression of a reporter protein.

A reporter protein is any protein that provides a phenotypic readout. Examples of reporter proteins include cell survival proteins, cell reproduction proteins, fluorescence proteins, bioluminescence proteins, enzymes that act on a substrate to produce a colorimetric signal, protein kinases, proteases, transcription factors, and regulatory proteins such as ubiquitin. The use of suitable reporter proteins in assays for determining PPIs is described, for example, in Wehr and Rossner (2016).

In these assays, activity of the reporter protein is controlled by the association of the first and second candidate protein. This can be achieved in several ways. For example, the reporter protein may be split into a first and second fragments of the reporter protein, such that the first and second fragments need to be brought into sufficient proximity (e.g. non-covalently interact) in order to reconstitute activity of the reporter protein. Reporter proteins that can be split into fragments in this way can be termed “split reporters”. Several split reporters are known in the art and include beta-lactamase, dihydrofolate reductase (DHFR), focal adhesion kinase (FAK), Gal4, GFP (split-GFP), horseradish peroxidase, infrared fluorescent protein IFP1.4, an engineered chromophore-binding domain (CBD), LacZ (beta-galactosidase), luciferase, TEV (Tobacco etch virus protease) and ubiquitin.

In some embodiments, the first candidate binding partner is linked (e.g. fused) to a first fragment of the reporter protein and the second candidate binding partner is linked (e.g. fused) to the second fragment of the reporter protein, where association (e.g. dimerisation) of the first and second candidate binding partners reconstitutes reporter protein activity. This assay may be termed the protein-fragment complementation assay, or PCA and is well known in the art. In cases where association of the first and second candidate binding partners reconstitutes reporter protein activity, the additional presence in the cell of a peptide that inhibits association between the first and second candidate binding partners will decrease activity of the reporter protein.

Other suitable assays may make use of a DNA-binding complex to inhibit or promote expression of the reporter protein as a way of controlling activity of the reporter protein. In these assays, the cell may further comprise a nucleic acid encoding the reporter protein. The nucleic acid encoding the reporter protein may be an expression cassette (also termed a “reporter protein expression cassette”), which may be delivered to the cell, optionally as part of an expression vector, or may be incorporated into the genome of the cell. The nucleic acid encoding the reporter protein comprises a binding site that the DNA-binding complex binds to and inhibits or promotes expression of the reporter protein. This DNA-binding based assay can be used in embodiments where the first and second candidate binding partners form the DNA-binding complex. Additionally, this DNA-binding based assay can be used in embodiments where the first and second candidate binding partners are linked to components that form a DNA-binding complex when brought into sufficient proximity (i.e. though association of the first and second candidate binding partners).

The DNA-binding complex may comprise any of the proteins of a particular bZIP family set forth in Table 1 above and the binding site in the nucleic acid encoding the reporter protein may the binding site associated with that bZIP family set forth in Table 1 above.

In some embodiments, one or more binding sites are located in the promoter or enhancer region of the nucleic acid encoding the reporter protein. In these embodiments, the DNA-binding complex typically has transcriptional activation or transcriptional repression activity such that upon binding to the binding site(s) it is capable of promoting or inhibiting expression of the reporter protein.

In other embodiments, one or more binding sites are located in the transcribed sequence (e.g. the coding sequence) of the nucleic acid encoding the reporter protein. In these embodiments, binding of the DNA-binding complex to the binding site(s) inhibits transcription of the reporter protein.

Accordingly in preferred embodiments, the cell comprises the first and second candidate binding partners and a nucleic acid encoding a reporter protein, where association of the first and second candidate binding partners form a DNA-binding complex that binds to one or more binding sites in the nucleic acid encoding the reporter protein such that binding of the DNA-binding complex to the binding site(s) inhibits expression of the reporter protein. In these embodiments an increase in expression of the reporter protein in the presence of the conformationally constrained peptide indicates that the conformationally constrained peptide is capable of inhibiting association of the first and second candidate binding partners.

Monitoring the activity and/or expression of the reporter protein will depend on the reporter protein used.

For example, where the reporter protein is a cell survival protein, then inhibition of expression and/or activity of the cell survival protein will result in cell death. Cell death can be determined by one of a number of techniques known to the person skilled in the art, e.g. the observing of morphological changes such as cytoplasmic blebbing, cell shrinkage, internucleosomal fragmentation and chromatin condensation. Use of a cell survival protein as a reporter protein can be advantageous as it gives a simple binary readout, i.e. the cell is either dead or alive. Methods using cell survival proteins as reporter proteins in screening for inhibitors that disrupt PPIs are known. See, for example, Park et al. (2007), which describes methods involving beta-lactamase in a fragmentation complementation strategy.

If the reporter protein is a cell reproduction protein, then inhibition of expression and/or activity of the cell reproduction protein will result in the cell being unable to proliferate and therefore unable to form progeny. Cell proliferation can be determined by one of a number of techniques known to the person skilled in the art, e.g. by counting of individual cells, foci or colonies, measuring metabolic activity using dyes such as MTT and WST-1, using nucleoside analogues such as bromodeoxyuridine (BrdU) and measuring incorporation of this analogue in the cells, staining dividing cells using reagents such as succinimidyl ester of carboxyfluorescein diacetate, and detecting proliferation markers such as PCNA, poisomerase IIB or phosphohistone H3. Inhibition of cell proliferation may also result in cell death, which can be measured as described above.

As a further example of a reporter protein that provides an observable phenotype, the reporter protein can be a fluorescent protein, a bioluminescent protein, or an enzyme that acts on a substrate to produce a colorimetric signal. In these cases, activity of the reporter proteins results in an observable signal when active that can therefore be monitored.

A conformationally constrained peptide that is able to modulate expression and/or activity of the reporter protein may be able to modulation expression and/or activity by at least 50%, by at least 2-fold, by at least 5-fold, or by at least 10-fold when compared to reporter protein expression and/or activity in an equivalent cell that lacks the conformationally constrained peptide. For example, where association of the first and second candidate binding partners results in a decrease in expression and/or activity of the reporter protein (e.g. a cell survival protein such as DHFR), an increase by at least 50% in expression and/or activity of the reporter protein (e.g. at least 50% more living cells) in the presence of the conformationally constrained peptide may indicate the constrained peptide is capable of modulating expression and/or activity of the reporter protein.

The conformationally constrained peptide may elicit a greater modulation (e.g. at least 50% greater modulation, at least 2-fold greater modulation, at least 5-fold greater modulation, or at least 10-fold greater modulation) of expression and/or activity of the reporter protein when compared to the ability of the un-crosslinked recombinant peptide to modulate expression and/or activity of the reporter protein.

This may indicate that conformationally constraining the peptide increases its ability to disrupt PPIs between the first and second candidate binding partner (e.g. the conformationally constrained peptide binds its target with a higher affinity). Thus, in some embodiments, the method further comprises determining whether the conformationally constrained peptide elicits greater modulation of the expression and/or activity of the reporter protein compared to the recombinant protein that lacks the thioether cross-links with the cross-linker. This may involve measuring the reporter protein expression and/or activity before and after the cell is contacted and cultured with the cross-linker.

Cross-Linkers

Suitable cross-linkers are known in the art for crosslinking cysteine (see for example: Fairlie & Dantas de Araujo, 2016 and Jo et al., 2012).

In some embodiments, the cross-linker is a compound of formula 1:

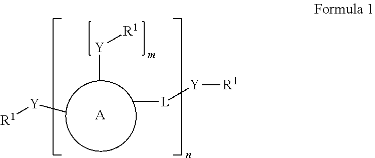

wherein
n is an integer selected from 1 to 3;
m is an integer selected from 0 to 2;
A is selected from C₂₋₆-alkenylene, C₅₋₁₂-arylene and C₅₋₁₂-heteroarylene;
Y is a covalent bond, C₁₋₆alkylene or —N(H)C(═O)CH₂—;
R¹ is selected from Cl, Br, I, or F; and
each L is independently selected from —C(═O)—, —C≡C—, —N═N—, C₁₋₆alkylene and a covalent bond.

As is understood in the art, if Y is a covalent bond and an L group attached to the Y is also a covalent bond, together these groups form a single covalent bond between A and R¹.

The R¹ groups provide reactive groups (e.g. leaving groups) for reaction with the cysteine. The A groups provide the linkers with structures suitable for conformationally constraining a peptide in a call when cross inked via the two derivatisable amino acid residues. For example, the A group may be conformational constrained into a geometry suitable for linking the two derivatisable amino acid residues.

In some embodiments A is selected from C₅₋₁₂-arylene and C₅₋₁₂-heteroarylene. For example, A may be selected from phenylene, pyridinylene, tetrazinylene or quinoxalinylene such a phenylene.

In some embodiments m is 0.

In some embodiments Y is methylene.

In some embodiments L is a covalent bond.

In some embodiments, R¹ is Br.

In some embodiments n is 1. In this way, the cross-linker can react with derivatisable amino acid residues at the i and i+3 or i and i+4 in the amino acid sequence of the recombinant peptide.

In some such embodiments A is selected from C₅₋₁₂-arylene and C₅₋₁₂-heteroarylene. For example, A may be selected from phenylene, pyridinylene, tetrazinylene or quinoxalinylene such a phenylene.

In some such embodiments m is 0.

In some such embodiments Y is a covalent bond or methylene, preferably Y is methylene.

In some such embodiments L is a covalent bond.

In some such embodiments, R¹ is Br, Cl or F, preferably R¹ is Br.

In some such embodiments A is selected from C₅₋₁₂-arylene and C₅₋₁₂-heteroarylene. For example, the cross-linker of formula 1 may be a compound of formula 2:

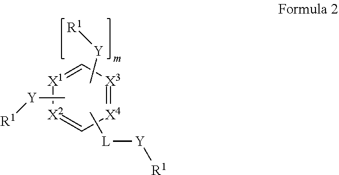

wherein each of R¹, Y, m and L is as defined above for formula 1; and
each of X¹, X², X³ and X⁴ are independently selected from N or CH.

In some embodiment of Formula 2, m is 0.

In some embodiment of Formula 2, Y is a covalent bond or methylene, preferably Y is methylene.

In some embodiment of Formula 2, L is a covalent bond.

In some embodiment of Formula 2, R¹ is Br, Cl or F, preferably R¹ is Br.

In some such embodiments A is C₅₋₁₂-arylene. For example, the cross-linker of formula 2 is a compound of formula 2a:

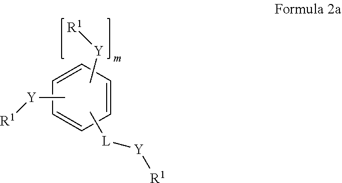

wherein each of R¹, Y, m and L is as defined above for formula 2.

In some such embodiments, the cross-linker of formula 2a is a compound of formula 2aa:

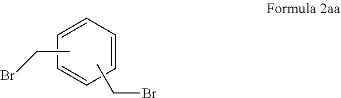

In other embodiments n is 2. In this way, the cross-linker can react with derivatisable amino acid residues at the i and i+7 or i and i+11 in the amino acid sequence of the recombinant peptide.

In some such embodiments A is selected from C₅₋₁₂-arylene and C₅₋₁₂-heteroarylene. For example, A may be selected from phenylene, pyridinylene, tetrazinylene or quinoxalinylene such a phenylene.

In some such embodiments m is 0.

In some such embodiments Y is methylene.

In some such embodiments L is a covalent, bond, —C(═O)—, —C≡C—, or —N═N—.

In some such embodiments, R¹ is Cl, I or Br.

In some such embodiments A is C₅₋₁₂-arylene. For example, the crosslinker of formula 1 is a compound of formula 3:

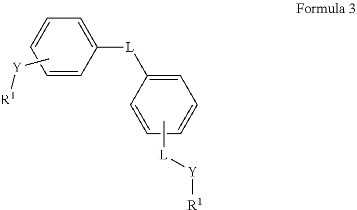

wherein each of R¹, Y, m and L is as defined above for formula 1.

In some embodiments of Formula 3, Y is methylene.

In some embodiments of Formula 3, L is a covalent bond, —C(═O)—, —C≡C—, or —N═N—.

In some embodiments of Formula 3, R¹ is Cl, I or Br.

In some embodiments the cross-linker may be selected from:

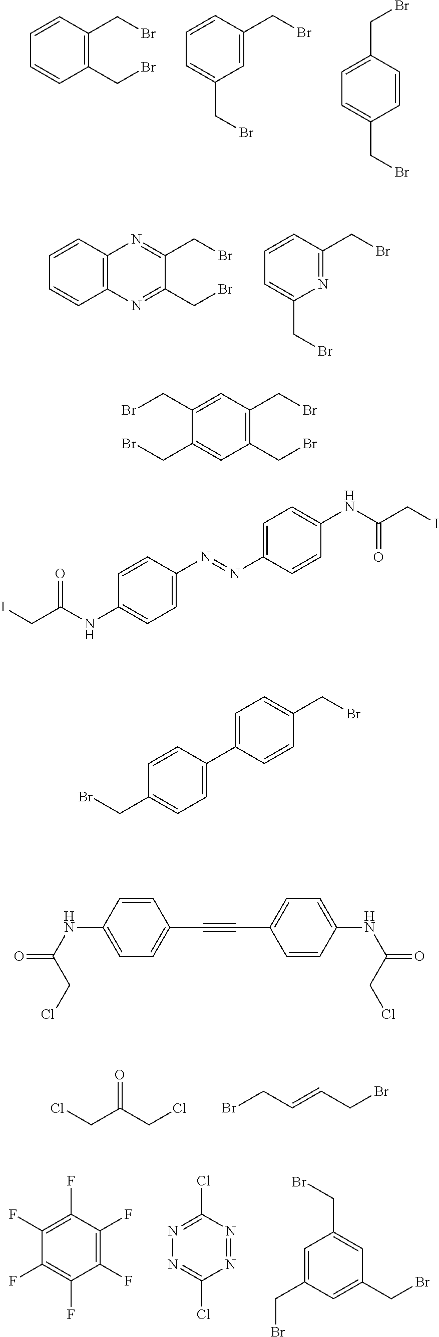

In some embodiments, the cross-linker is a compound of formula 2a selected from:

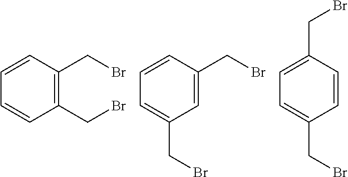

Preferably, the cross linker is:

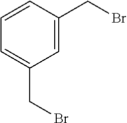

The term alkylene as used herein refers to a saturated, branched, or straight chain hydrocarbon group having two monovalent radical centres derived by the removal of two hydrogen atoms from the same or two different carbon atoms of a parent alkane. The number of carbon atoms in the alkylene group may be specified using the above notation, for example, when there are from 1 to 6 carbon atoms the term “C₁₋₆-alkylene” may be used. Alkylene groups may be optionally substituted. Example alkylene groups include methylene (—CH₂—).

The term alkenylene as used herein refers to a linear or branched-chain hydrocarbon group having two monovalent radical centres derived by the removal of two hydrogen atoms from the same or two different carbon atoms with at least one site of unsaturation, i.e., a carbon-carbon double bond. The alkenylene radical may be optionally substituted, and includes radicals having “cis” and “trans” orientations, or alternatively, “E” and “Z” orientations. The number of carbon atoms in the alkenylene group may be specified using the above notation, for example, when there are from 2 to 6 carbon atoms the term “C₂₋₆-alkenylene” may be used. Alkenylene groups may be optionally substituted. Example alkenylene groups include, but are not limited to, ethenylene (—CH═CH—), prop-1-enylene (—CH═CHCH₂—).

The term arylene as used herein refers to a carbocyclic aromatic radical group having two monovalent radical centres derived by the removal of two hydrogen atoms from the same or two different carbon atoms. Arylene includes groups having a single ring and groups having more than one ring such a fused rings or spirocycles. In the case of groups having more than one ring, at least one of the rings is aromatic. The number of carbon atoms in the arylene group may be specified using the following notation, for example, when there are from 5 to 12 carbon atoms the term “C₅₋₁₂-arylene” may be used.

Arylene groups may be optionally substituted, for example they may be optionally substituted by one or more halogen atoms such as fluorine. Examples of arylene groups include phenylene, naphthylene.

The term heteroarylene as used herein refers to an aromatic radical group with heteroatoms in the aromatic ring having two monovalent radical centres derived by the removal of two hydrogen atoms from the same or two different atoms. Suitable heteroatoms include N and S. Heteroarylene includes groups having a single ring and groups having more than one ring such a fused rings or spirocycles. In the case of groups having more than one ring, at least one of the rings is aromatic. The number of atoms (carbon atoms plus any heteroatoms) in the heteroarylene group may be specified using the following notation, for example, when there are from 5 to 12 carbon atoms plus heteroatoms making up the ring structure, the term “C₅₋₁₂-heteroarylene” may be used. Heteroarylene groups may be optionally substituted. Examples of heteroarylene groups include pyridinylene (derived from pyridine), tetrazinylene (derived from terazine), and quinoxazinylene (derived from quinoxazine).

Where a chemical formula, such as a ring, is shown with substituents attached and the substituents are not attached at a specific location, as is common practice, the substituents may be attached at any suitable position. For example in formula 2aa:

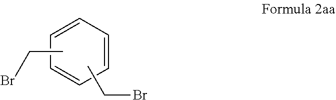

The two —CH₂Br groups may be attached in any combination of positions on the phenyl ring e.g. formula 2aa covers 1,2 dibromomethylbenzene, 1,3 dibromomethylbenzene, and 1,4 dibromomethylbenzene

Cells and Culture Conditions

The methods of the invention functions in live cells, i.e. the methods are performed in cellulo unless the context clearly dictates otherwise. The term “in cellulo” is intended to encompass experiments that take place involving cells and may be on cultured cells or may be on cells or tissues that have been taken from an organism. The methods of the invention are not practiced on the human or animal body.

Any cell suitable for the expression of recombinant peptides may be used for the screening method described herein. The cell may be a prokaryote or eukaryote. Typically the cells are isolated cells.

The cell used in the method may be a bacterial cell, such as a gram negative bacterial cell. In some embodiments, the bacterial cell is an Escherichia coli cell, for example BL21 (DE3), XL-1, RV308, or DH5alpha cells. Methods where the cell is a bacterial cell may involve culturing the bacterial cell in suitable media. Such techniques are well known to those of skill in the art.

Alternatively, the cell is a eukaryotic cell such as a yeast cell, a plant cell, insect cell or a mammalian cell.

In some embodiments, the cell is a mammalian cell, for example a human cell. Mammalian cells, especially human cells, may be somatic cells. Screening methods where the cell is a eukaryotic cell may involve culture or fermentation of the eukaryotic cell. The culture or fermentation may be performed in a bioreactor provided with an appropriate supply of nutrients, air/oxygen and/or growth factors. Culture, fermentation and separation techniques are well known to those of skill in the art.

The method involves contacting and culturing the cell in the presence of the cross-linker. For example, the cross-linker may be present in or added to the culture media (e.g. a solid, liquid or semi-solid media containing components such as nutrients and antibiotics to support cell growth) that the cell is being cultured in, wherein the cross-linker is capable of permeating the cell such that it moves from the media into the cytosol of the cell (i.e. through the cell membrane, and cell wall if present) where it can react with the reactive thiol groups present in the intracellularly-localised recombinant protein. If the cells are being grown on a solid growth medium such as an agar plate, the cross-linker may be included in the solid growth medium. If the cells are being grown in a liquid medium, the cross-linker may be included or added to the liquid medium.

The cell may be cultured in the presence of the cross-linker for a period of at least 20 minutes, at least 30 minutes, at least 1 hour, at least 2 hours, at least 6 hours, at least 12 hours, at least 24 hours, at least 36 hours, or at least 48 hours. In some embodiments the cell is kept in the dark while it is being cultured in the presence of the cross-linker. This may be useful, for example, to reduce the likelihood of any oxidation occurring.

The concentration of cross-linker included in the culture media may be between 5 μM to 1 mM. In some embodiments, the concentration of cross-linker is present at a concentration of between 5 μM and 500 μM, between 5 μM and 100 μM

In some embodiments, the cell is cultured in the presence of the cross-linker at a pH of between about pH 7 and about pH 9. In some embodiments, the pH may be between about pH 7.5 and about pH 8, preferably about pH 8. Methods of adjusting the pH of the culture media using, e.g. buffers, are known in the art. As explained in Jo et al. 2012, certain cross-linkers such as DBMB operate under mild conditions where the pH is about 7.5 to 8.

In some embodiments, the cell may be cultured in the presence of a reducing agent in addition to the cross-linker. Examples of suitable reducing agents include tris(2-carboxyethyl) phosphine (TCEP), dithiothreitol (DTT), 2-mercaptoethanol, and 2-mercaptothylamine. In some embodiments, the cell is cultured in the presence of TCEP.

Inhibitors and Kits

In some embodiments, the methods described herein further comprise isolating the conformationally constrained peptide that has been identified as being able to inhibit association between a first and second candidate binding partner (e.g. a conformationally constrained peptide that is able to modulate expression and/or activity of a reporter protein). Isolated conformationally constrained peptides identified by the methods of the present invention, as well as the nucleic acids encoding them, therefore form further aspects of the present invention.

In another aspect, the present invention provides a kit comprising:
i) a nucleic acid encoding a recombinant peptide, wherein the recombinant peptide comprises at least two derivatisable amino acid residues located at anchoring positions, each derivatisable amino acid comprising a reactive thiol group; and
ii) a cross-linker, wherein the cross-linker is capable of reacting with said reactive thiol groups.

The recombinant peptide and cross-linker in the kit may be as defined above.

The kit may further comprise one or more nucleic acids encoding the first and second candidate binding partners and/or the reporter protein, which may be as defined above.

The kit may further comprise a cell for expressing the recombinant protein. The cell may be as defined above.

The invention includes the combination of the aspects and preferred features described except where such a combination is clearly impermissible or expressly avoided.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### SUMMARY OF THE FIGURES

Embodiments and experiments illustrating the principles of the invention will now be discussed with reference to the accompanying figures in which:

FIG. 1 illustrates a representative spectrum comparing Ellman's assay signal before (pre-reaction) and after (post-reaction) the DBMB cross-linker is added in an in vitro reaction.

FIG. 2 provides absorbance scans carried out at 200-300 nm on 1) DBMB alone; 2) two purified SEC fractions isolated from cells grown in the absence of DBMB (‘no DBMB’); and 3) two purified size exclusion chromatography (SEC) fractions isolated from cells cultured in the presence of DBMB. As expected due to the aromatic group present in DBMB, the sample containing DBMB alone resulted in a very high absorbance profile (>1 units). No significant peak was observed for the peptide isolated from cells grown in the absence of DBMB. In contrast peptides isolated from cells grown in the presence of DBMB lead to a significant increase in absorption, providing evidence that DBMB formed cross-links with the cysteine residues in this peptide.

### EXAMPLES

### Example 1—In Vitro Cross-Linking Using DBMB

An experiment was carried out to confirm that DBMB could be used in an in vitro experiment to cross-link cysteine residues present in a peptide.

Cysteine peptide CLIPS in vitro reaction—A previously identified inhibitor of AP-1, FosW (Mason et al., 2006), was used to generate recombinant peptides having cysteines located at i and i+4 positions in the peptide. The FosW peptide contains multiple heptad repeats, each having the motif labelled as abcdefg.

Four peptides were generated: Hep1 containing cysteines at b and fin the first heptad repeat; Hep2 containing cysteins at the each comprising Four peptides were generated, labelled Hep1, Hep2, Hep3 and Hep4 and contained cysteines located at positions b. Reaction of 1,3-bisbromomethylbenzene (α,α′-Dibromo-m-xylene) with cysteine peptides was achieved through optimisation of a published protocol (Timmerman et al., 2005). Generally, 100 μM peptide in 500 μl 75% water:25% acetonitrile (Fisher), 20 mM ammonium bicarbonate (in water), 5 equivalents 1,3-bisbromomethylbenzene (in acetonitrile), and 2 equivalents TCEP hydrochloride (in water) was reacted at pH8 and room temperature for approx. 4.5 hours in the dark.

Peptide Characterisation—Ellman's reagent 5,5′-dithiobis(2-nitrobenzoic acid; DTNB) (Sigma) was used to indicate either 0,1, or 2 free thiols prior to high performance liquid chromatography (HPLC) and mass spectrometry (MS). Briefly, 4.5 or 9 μM peptide was added to 150 μM Ellman's reagent in 100 mM sodium phosphate buffer (Sigma) containing 0.1 M EDTA (Sigma) and absorbance monitored at 412 nm using a UV spectrophotometer (Cary 50) with a 1 cm pathlength.

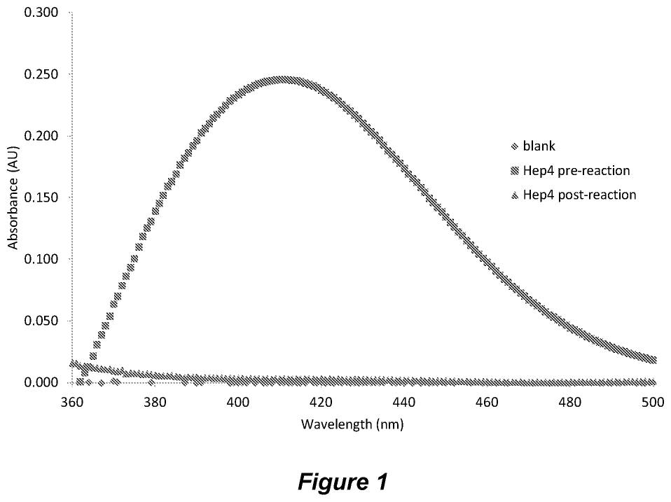

Results of the assay demonstrated that the DTNB signal was either 0, 1, or 2 molar equivalents, providing evidence that the bis-acylation had worked correctly. A representative spectrum of the peptide pre- and post-crosslinking is provided in FIG. 1. As illustrated in this representative spectrum, no free thiols were observed in the peptide following cross-linking.

Following HPLC, a small scale electrospray mass spectrometry experiment revealed that all peptides returned spectra displaying the expected mass increase of ˜102 Da following DBMB cross-linking. The expected ˜102 Da increase was calculated from the mass of 1,3-bisbromomethylbenzene (263.96 Da) without the two bromine atoms and two peptide sulfhydryl hydrogen atoms. The results from the mass spectrometry experiment are provided in Table 2 as follows:

TABLE 2

| | Un-reacted mass | Reacted mass | Mass difference |
|---|---|---|---|
| Species | (Da) | (Da) | (Da) |
| Hep1 | 4372.29 | 4474.34 | 102.05 |
| Hep2 | 4300.24 | 4402.29 | 102.05 |
| Hep3 | 4267.19 | 4369.24 | 102.05 |

### Example 2—in Cell Cross-Linking Using DBMB

Having confirmed that DBMB could be used to cross-link cysteines in an in vitro cross-linking reaction, an experiment was designed to establish whether this reagent could be used to cross-link cysteines in peptides localised within cells.

A previously identified inhibitor of AP-1, FosW (Mason et al., 2006), was used to generate recombinant peptides having cysteines located at i and i+4 positions in the peptide. Nucleic acids encoding recombinant peptides having the sequences set out in Table 3 were generated (locations of the introduced cysteines are emphasised):

TABLE 3

| Name | Amino acid sequence |
|---|---|
| Fos cyst1 | ASLCELQCEIEQLEERNYALRKEIEDLQKQLEKLGAP |
| Fos cyst2 | ASLDELQAEI [symbol] QLE [symbol] RNYALRKEIEDLQKQLEKLGAP |
| Fos cyst3 | ASLDELQAEIEQLEERN [symbol] ALR [symbol] EIEDLQKQLEKLGAP |
| Fos cyst4 | ASLDELQAEIEQLEERNYALRKEI [symbol] DLQ [symbol] QLEKLGAP |
| Fos cyst5 | ASLDELQAEIEQLEERNYALRKEIEDLQ [symbol] QLE [symbol] LGAP |

Nucleic acids encoding these peptides were inserted into the p230d plasmid and this was confirmed by sequencing. The peptides were His-tagged.

In order to confirm that DBMB is not toxic to E. coli, an experiment was designed to determine whether these cells can grow in the presence of different concentrations of DBMB. A stock solution of 100 mM DBMB was prepared in methanol and liquid broth (LB) plates were prepared by adding different concentration of DBMB, 5 μM, 10 μM, 15 μM, 20 μM and 40 μM.

Both Fos cyst1 and Fos cyst3 were transformed into BL21 Gold and plated on LB (Amp, 20-40 μM DBMB, pH 8.0), incubated at 37° C. overnight. Both plates showed good growth, confirming DBMB is not toxic at this level and can be used with protein expression.

Having confirmed that DBMB is not toxic to E. coli cells at these concentrations, cells were transformed with these peptides and cultured in the presence of 40 μM DBMB. 1 litre cultures expressing the peptide were grown in the presence of 40 μM DBMB until reaching OD₆₀₀ of 0.6-0.8, at which point the cells were spun down and lysed. The soluble and insoluble fractions were isolated and the presence of the recombinant peptide in the soluble fraction confirmed by SDS-PAGE (data not shown). This demonstrates that the peptide was expressed and localised to the cytosol of the cells.

The His-tagged peptides were purified from the soluble fraction by affinity chromatography (using a nickel resin) and then by size exclusion chromatography (SEC) using standard techniques. The purified peptide analysed by carrying out absorbance scans between 200-300 nm using a 1 cm quartz cell in a Cary 50 Spectrophotometer (Varian). Since DBMB contains an aromatic ring that absorbs UV light within this range, a purified peptide that contains DBMB cross-linked with the cysteine residues in the peptide should result in an increase in absorption at this range

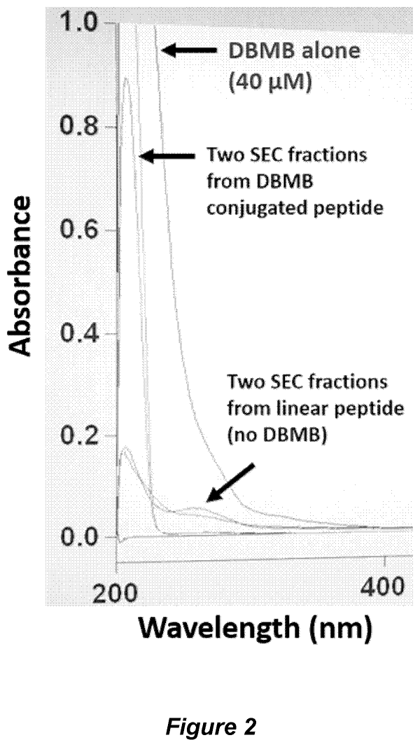

The absorbance scans for two SEC fractions of purified Fos cyst3 peptide (SEQ ID NO: 4) isolated from cells incubated in the presence of 40 μM DBMB were compared with the absorbance scans of DBMB alone (40 μM) and two SEC fractions of purified Fos cyst3 peptide isolated from cells grown in the absence of DBMB. Representative absorbance scans are provided in FIG. 2.

The results from these absorbance scans revealed a significant increase in absorption for the peptide purified from cells grown in the presence of DBMB whereas no significant peak was observed for a peptide isolated from cells grown in the absence of DBMB. This increase in absorption provides evidence that DBMB is capable of moving from the culture media into the cytosol of the cells, where it forms cross-links with cysteine residues within peptides expressed in those cells.

Further confirmation that DBMB is able to cross-link the cysteines in the peptide whilst present in the cell is provided using HPLC, Ellman's assay, mass spectrometry and/or circular dichroism (CD) assays. A brief explanation of the use of these exemplary assays to demonstrate DBMB cross-linking is provided as follows:

HPLC. Following SEC purification peptides are analysed by HPLC using a C18 peptide semi-prep column to establish that the DBMB leads to changes in hydrophobicity and therefore elution profiles.

DTNB (Ellman's reagent). Ellmans reagent is used to indicate either 0,1, or 2 free thiols prior to HPLC and MS. Moreover the assay is also used to demonstrate that the DTNB signal was either 0, 1, or 2 molar equivalents, providing evidence that the bis-acylation works correctly. Free thiols are assayed via reaction with Ellman's reagent 5,5′-dithiobis(2-nitrobenzoic acid; DTNB) (Sigma) and monitoring absorbance at 412 nm using 4.5 or 9 μM peptide and 150 μM Ellman's reagent, in 100 mM sodium phosphate buffer (Sigma) containing 0.1 M EDTA (Sigma) with a 1 cm pathlength on a UV spectrophotometer (Cary 50).

Electrospray Mass spectrometry. Following HPLC, a small scale reaction reveals correct reacted mass as illustrated in Table 2, again demonstrating that only intra-molecular xylene thioester bridging had occurred and that only the expected mass increase of 102 Da is observed.

CD. Peptides are analysed using a Chirascaninstrument (Applied Photophysics), recording the ellipticities of linear peptide (no DBMB) or constrained peptide (purified from cells grown in the presence of DBMB at a total peptide concentration (Pt) of 20 μM dissolved in 10 mM potassium phosphate buffer with 100 mM potassium fluoride (pH 7).

### REFERENCES

A number of publications are cited above in order to more fully describe and disclose the invention and the state of the art to which the invention pertains. Full citations for these references are provided below. The entirety of each of these references is incorporated herein.
Azzarito, V., Long, K., Murphy, N. S., & Wilson, A. J. (2013). Inhibition of α-helix-mediated protein-protein interactions using designed molecules. Nature chemistry, 5(3), 161-173. https://doi.org/10.1038/nchem.1568
Baxter, D., Perry, S. R., Hill, T. A., Kok, W. M., Zaccai, N. R., Brady, R. L., Fairlie, D. P., & Mason, J. M. (2017). Downsizing Proto-oncogene cFos to Short Helix-Constrained Peptides That Bind Jun. ACS chemical biology, 12(8), 2051-2061. https://doi.org/10.1021/acschembio.7b00303
Craik D. J. (2012). Protein folding: Turbo-charged crosslinking. Nature chemistry, 4(8), 600-602. https://doi.org/10.1038/nchem.1417
Heinis, C., & Winter, G. (2015). Encoded libraries of chemically modified peptides. Current opinion in chemical biology, 26, 89-98. https://doi.org/10.1016/j.cbpa.2015.02.008
Fairlie, D. P., & Dantas de Araujo, A. (2016). Review stapling peptides using cysteine crosslinking. Biopolymers, 106(6), 843-852. https://doi.org/10.1002/bip.22877
Jo, H., Meinhardt, N., Wu, Y., Kulkarni, S., Hu, X., Low, K. E., Davies, P. L., DeGrado, W. F., & Greenbaum, D. C. (2012). Development of α-helical calpain probes by mimicking a natural protein-protein interaction. Journal of the American Chemical Society, 134(42), 17704-17713. https://doi.org/10.1021/ja307599z
Jochim, A. L., & Arora, P. S. (2010). Systematic analysis of helical protein interfaces reveals targets for synthetic inhibitors. ACS chemical biology, 5(10), 919-923. https://doi.org/10.1021/cb1001747
Mason, J. M., Schmitz, M. A., Müller, K. M., & Arndt, K. M. (2006). Semirational design of Jun-Fos coiled coils with increased affinity: Universal implications for leucine zipper prediction and design. Proceedings of the National Academy of Sciences of the United States of America, 103(24), 8989-8994. https://doi.org/10.1073/pnas.0509880103
Mern, D. S., Hasskarl, J., & Burwinkel, B. (2010). Inhibition of Id proteins by a peptide aptamer induces cell-cycle arrest and apoptosis in ovarian cancer cells. British journal of cancer, 103(8), 1237-1244. https://doi.org/10.1038/sj.bjc.6605897
Park, J. H., Back, J. H., Hahm, S. H., Shim, H. Y., Park, M. J., Ko, S. I., & Han, Y. S. (2007). Bacterial beta-lactamase fragmentation complementation strategy can be used as a method for identifying interacting protein pairs. Journal of microbiology and biotechnology, 17(10), 1607-1615.
Raj, M., Bullock, B. N., & Arora, P. S. (2013). Plucking the high hanging fruit: a systematic approach for targeting protein-protein interactions. Bioorganic & medicinal chemistry, 21(14), 4051-4057. https://doi.org/10.1016/j.bmc.2012.11.023
Rao, T., Ruiz-Gómez, G., Hill, T. A., Hoang, H. N., Fairlie, D. P., & Mason, J. M. (2013). Truncated and helix-constrained peptides with high affinity and specificity for the cFos coiled-coil of AP-1. PloS one, 8(3), e59415. https://doi.org/10.1371/journal.pone.0059415
Robertson, N. S., & Spring, D. R. (2018). Using Peptidomimetics and Constrained Peptides as Valuable Tools for Inhibiting Protein-Protein Interactions. Molecules (Basel, Switzerland), 23(4), 959. https://doi.org/10.3390/molecules23040959
Timmerman, P., Beld, J., Puijk, W. C., & Meloen, R. H. (2005). Rapid and quantitative cyclization of multiple peptide loops onto synthetic scaffolds for structural mimicry of protein surfaces. Chembiochem: a European journal of chemical biology, 6(5), 821-824. https://doi.org/10.1002/cbic.200400374
Yoo, D. Y., Hauser, A. D., Joy, S. T., Bar-Sagi, D., & Arora, P. S. (2020). Covalent Targeting of Ras G12C by Rationally Designed Peptidomimetics. ACS chemical biology, 15(6), 1604-1612. https://doi.org/10.1021/acschembio.0c00204

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

Sequence Annex
Amino acid sequence of FosW peptide
(SEQ ID NO: 1)
ASLDELQAEIEQLEERNYALRKEIEDLQKQLEKLGAP
Amino acid sequence of Fos cyst1 exemplary
recombinant peptide
(SEQ ID NO: 2)
ASLCELQCEIEQLEERNYALRKEIEDLQKQLEKLGAP
Amino acid sequence of Fos cyst2 exemplary
recombinant peptide
(SEQ ID NO: 3)
ASLDELQAEICQLECRNYALRKEIEDLQKQLEKLGAP
Amino acid sequence of Fos cyst3 exemplary
recombinant peptide
(SEQ ID NO: 4)
ASLDELQAEIEQLEERNCALRCEIEDLQKQLEKLGAP
Amino acid sequence of Fos cyst4 exemplary
recombinant peptide
(SEQ ID NO: 5)
ASLDELQAEIEQLEERNYALRKEICDLQCQLEKLGAP
Amino acid sequence of Fos cyst5 exemplary
recombinant peptide
(SEQ ID NO: 6)
ASLDELQAEIEQLEERNYALRKEIEDLQCQLECLGAP

1. A method of producing a conformationally constrained peptide in a cell, the method comprising:
i) providing a cell containing a recombinant peptide, wherein the recombinant peptide comprises at least two derivatisable amino acid residues located at anchoring positions, each derivatisable amino acid comprising a reactive thiol group;
ii) contacting the cell with a cross-linker, wherein the cross-linker is capable of reacting with said reactive thiol groups; and
iii) culturing the cell in the presence of the cross-linker, such that the cross-linker forms thioether cross-links with the at least two derivatisable amino acids, thereby producing the conformationally constrained peptide in the cell.

2. A method according to claim 1, wherein providing the cell containing the recombinant peptide comprises delivering a nucleic acid encoding the recombinant peptide to the cell, such that the cell expresses the recombinant peptide.

3. A method according to claim 1 or claim 2, wherein the cell further comprises first and second candidate binding partners and the method further comprises assaying whether the conformationally constrained peptide is able to inhibit association between the first and second candidate binding partner.

4. A method according to claim 3, wherein assaying for whether the conformationally constrained peptide is able to inhibit association between the first and second candidate binding partners comprises determining whether the conformationally constrained peptide is able to modulate expression and/or activity of a reporter protein.

5. A method according to claim 4, wherein:
i) the first candidate binding partner is linked to a first fragment of the reporter protein and the second candidate binding partner is linked to the second fragment of the reporter protein, wherein association of the first and second candidate binding partners reconstitutes reporter protein activity; or
ii) association of the first and second candidate binding partners forms a DNA-binding complex that binds a binding site in a nucleic acid encoding the reporter protein, wherein binding to the binding site inhibits or promotes expression of the reporter protein.

6. A method according to any one of the preceding claims, wherein the anchoring positions are positions i and i+3, i and i+4, i and i+7, or i and i+11 in the amino acid sequence of the recombinant peptide, optionally wherein the conformationally constrained peptide is a helix-constrained peptide.

7. A method according to any one of the preceding claims, wherein the only amino acid residues comprising a reactive thiol group in the recombinant peptide are those located at the anchoring positions.

8. A method according to any one of the preceding claims, wherein the at least two derivatisable amino acid residues are cysteine residues.

9. A method according to any one of the preceding claims, wherein the cross-linker is a compound of formula 1: 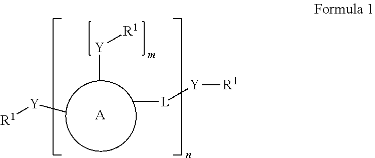
wherein
n is an integer selected from 1 to 3;
m is an integer selected from 0 to 2;
A is selected from C₂₋₆-alkenylene, C₅₋₁₂-arylene and C₅₋₁₂-heteroarylene;
Y is a covalent bond, C₁₋₆-alkylene or —N(H)C(═O)CH₂—;
R¹ is selected from Cl, Br, I, or F; and
each L is independently selected from —C(═O)—, —C≡C—, —N═N—, C₁₋₆-alkylene and a covalent bond.

10. A method according to claim 9, wherein the cross-linker is a compound of formula 2: 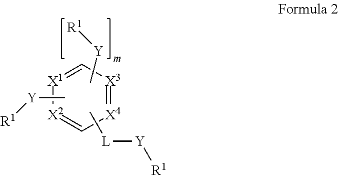
each of X¹, X², X³ and X⁴ are independently selected from N or CH.

11. A method according to claim 9 or claim 10, wherein the cross-linker is a compound of formula 2a:

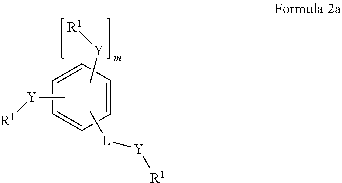

12. A method according to any one of the preceding claims, wherein the, the cross-linker is 1,3 dibromomethylbenzene (DBMB) having the formula:

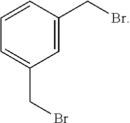

13. A method according to any one of the preceding claims, wherein the concentration of the cross-linker is between 1 μM and 1 mM, optionally between 10 μM and 100 μM.

14. A method according to any one of the preceding claims, wherein the cell is cultured in the presence of the cross-linker for a period of at least 20 minutes.

15. A method according to any one of the preceding claims, wherein the recombinant peptide is derived from the FosW peptide having the amino acid sequence set forth in SEQ ID NO: 1 (AASLDELQAEIEQLEERNYALRKEIEDLQKQLEKLGAP, FosW), modified to comprise at least two derivatizable amino acids located at anchoring positions in the FosW peptide.

16. A method according to any one of the preceding claims, wherein the step iii) is carried out at between about pH 7 and about pH 9, optionally at about pH 8.

17. A method according to any one of the preceding claims, further comprising culturing the cell in the presence of tris(2-carboxyethyl) phosphine (TCEP).

18. A method according to any one of the preceding claims, wherein the cell is a bacterial cell, optionally an E. coli cell.

19. A method according to any one of claims 1 to 17, wherein the cell is a eukaryotic cell, optionally a human cell.

20. A method according to any one of the preceding claims, the method further comprising isolating the conformationally constrained peptide from the cell.

21. A kit comprising:
i) a nucleic acid encoding a recombinant peptide, wherein the recombinant peptide comprises at least two derivatisable amino acid residues located at anchoring positions, each derivatisable amino acid comprising a reactive thiol group; and
ii) a cross-linker, wherein the cross-linker is capable of reacting with said reactive thiol groups, such that the cross-linker is capable of forming thioether cross-links with the at least two derivatisable amino acids.

22. A kit according to claim 21, wherein the, the cross-linker is 1,3 dibromomethylbenzene (DBMB) having the formula:

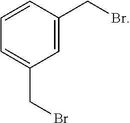

23. A kit according to claim 21 or claim 22, the kit further comprising a cell for expressing the recombinant peptide, optionally wherein the cell is a bacterial cell, such as an E. coli cell.


SEQUENCE LISTING (9 sequences)

SEQ ID NO: 1 - protein, 37 aa, Artificial Sequence
  synthetic construct
ASLDELQAEIEQLEERNYALRKEIEDLQKQLEKLGAP

SEQ ID NO: 2 - protein, 37 aa, Artificial Sequence
  synthetic construct
ASLCELQCEIEQLEERNYALRKEIEDLQKQLEKLGAP

SEQ ID NO: 3 - protein, 37 aa, Artificial Sequence
  synthetic construct
ASLDELQAEICQLECRNYALRKEIEDLQKQLEKLGAP

SEQ ID NO: 4 - protein, 37 aa, Artificial Sequence
  synthetic construct
ASLDELQAEIEQLEERNCALRCEIEDLQKQLEKLGAP

SEQ ID NO: 5 - protein, 37 aa, Artificial Sequence
  synthetic construct
ASLDELQAEIEQLEERNYALRKEICDLQCQLEKLGAP

SEQ ID NO: 6 - protein, 37 aa, Artificial Sequence
  synthetic construct
ASLDELQAEIEQLEERNYALRKEIEDLQCQLECLGAP

SEQ ID NO: 7 - DNA, 13 bp, Artificial Sequence
  synthetic construct
  misc_feature (7)..(7): n = G or C
tgctgantcagca

SEQ ID NO: 8 - DNA, 15 bp, Artificial Sequence
  synthetic construct
tgctgagcgtcagca

SEQ ID NO: 9 - protein, 38 aa, Artificial Sequence
  synthetic construct
AASLDELQAEIEQLEERNYALRKEIEDLQKQLEKLGAP